(19) United States
(12) Patent Application Publication
(10) Pub. No.: US 2008/0199412 A1
(43) Pub. Date: Aug. 21, 2008

(54) ANTI-STAINING ANTIBACTERIAL DENTIFRICE

(75) Inventors: Nebojsa Milanovich, Somerset, NJ (US); Abdul Gaffar, Princeton, NJ (US); Michael Prencipe, West Windsor, NJ (US); Thomas Boyd, Metuchen, NJ (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, NEW YORK, NY (US)

(21) Appl. No.: 12/100,922

(22) Filed: Apr. 10, 2008

Related U.S. Application Data
(63) Continuation of application No. 10/859,264, filed on Jun. 2, 2004.

(51) Int. Cl. A61K 8/55; A61K 8/69; A61Q 11/00

(52) U.S. Cl. 424/49; 424/52

(57) ABSTRACT

An anti-staining, antibacterial dentifrice comprises an anti-staining effective amount of an orally acceptable polymer or copolymer that comprises a plurality of monomeric groups of formula (I)

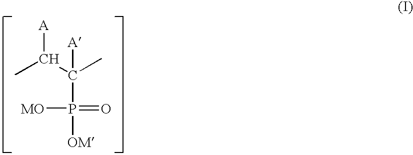

wherein (a) one of A and A′ is hydrogen and the other is a moiety (X)ₙ(R)ₘ, (b) n in individual such moieties is independently 0 or 1, (c) linking groups X if present independently comprise an oxygen, sulfur, nitrogen, phosphorus or silicon atom, (d) where n is 0, m is 1, and where n is 1, m is independently an integer from 1 to 3 as determined by X, (e) terminal groups R are independently hydrogen or C₁₋₁₈ organic radicals, and (t) M and M′ are independently selected from hydrogen, alkali metal and ammonium; said polymer or copolymer having an average molecular weight of at least about 1,000. The dentifrice further comprises an antibacterial effective amount of an orally acceptable halogenated diphenylether antibacterial agent, and an antibacterial enhancing effective amount of an orally acceptable polyvinylmethylether/maleic anhydride copolymer.

This application is a continuation of U.S. application Ser. No. 10/859,264, filed Jun. 2, 2004.

### FIELD

This invention relates to methods and compositions for dental care, more particularly to such methods and compositions having useful anti-staining as well as antibacterial activity.

### BACKGROUND

It is known to provide, as ingredients of an antibacterial, e.g., anti-plaque, dentifrice, a halogenated diphenylether antibacterial agent such as triclosan (2,4,4′-trichloro-2′-hydroxydiphenylether) in an anti-plaque effective amount, and a synthetic anionic polymeric polycarboxylate. in particular a polyvinylmethylether/maleic anhydride (PVME/MA) copolymer such as those available under the Gantrez™ brand of ISP, Wayne, N.J. See for example U.S. Pat. No. 4,894,220 to Nabi & Gaffar, wherein it is stated that an synthetic anionic polymeric polycarboxylate is effective to enhance delivery to and retention on a dental surface of an antibacterial agent.

It would be desirable to combine in a single dentifrice a halogenated diphenylether antibacterial agent, as enhanced using a PVME/MA copolymer, with an agent to prevent chemical staining of teeth. Polyphosphates such as sodium hexametaphosphate are known to be effective in chemical stain prevention (see Baig et al. (2002), J. Clin. Deit., 13(1), 19-24). However, a difficulty arises in coformulating a polyphosphate with a halogenated diphenylether such as triclosan because of chemical incompatibility of these ingredients, leading to loss of antibacterial efficacy.

One approach might be to provide a dual-component dentifrice having, for example, a halogenated diphenylether antibacterial agent and a PVME/MA copolymer in a first component and a polyphosphate stain prevention agent in a second component of the dentifrice. The two components, the first giving antibacterial, e.g., anti-plaque, benefit and the second giving anti-staining benefit, can be kept physically separate until dispensed for use. However, to minimize cost and inconvenience. it is generally preferable to formulate all desired ingredients in a single homogeneous multi-benefit composition.

Accordingly, a single-component dental care composition providing both antibacterial and anti-staining efficacy would represent a useful advance in the art, provided the anti-staining agent can be shown not to adversely affect antibacterial activity of the antibacterial agent or agents.

It is known to combine in a single-component dentifrice a halogenated diphenylether antibacterial agent such as triclosan with a phosphonic acid polymer. For example, U.S. Pat. No. 5,032,386 to Gaffar et al. states that such polymers, including for example poly(1-phosphonopropene) and poly(β-styrene phosphonic acid) can enhance delivery of an antibacterial agent to oral surfaces.

U.S. Pat. No. 5,296,214 to Gaffar discloses polyvinylphosphonates having an average molecular weight of about 1,000 to about 1,000,000 as ingredients of oral care products said to enhance delivery of an antibacterial agent to oral surfaces.

Polyvinylphosphonates have been further disclosed as inhibitors of salivary hydrolysis of polyphosphate anticalculus agents (see, for example, U.S. Pat. No. 5,094,844 to Gaffar et al.).

Polyvinylphosphonates have been still further disclosed as anticalculus agents per se (see, for example, U.S. Pat. No. 3,429,963 to Shedlovsky).

A method of inhibiting dental plaque and gingivitis, using a composition comprising a polyvinylphosphonate having a number average molecular weight of about 4,000 to 9,100, was proposed in U.S. Pat. No. 4,816,245 to Gaffar.

It is reported in British Patent No. 1 372 199 of Colgate-Palmolive Company that polyethylene monosodium polyphosphonate having on average one phosphonate group for every 6-7 carbon atoms on the polyethylene chain “is strongly absorbed onto tooth enamel”, resulting in inhibition of bacterial adhesion and growth on treated surfaces. Other “suitable materials” are said to include “homopolymeric sodium vinyl phosphonate (M.W. 20,000)”.

U.S. Pat. No. 6,509,007 to Rajaiah et al. discloses an oral care composition comprising polybutene and one or a combination of “oral care actives” that can include an anticalculus agent, e.g., a polyvinylphosphonate, and/or an antibacterial agent, e.g., triclosan. Patents and publications cited above are incorporated herein by reference.

### SUMMARY

It has now surprisingly been discovered that certain polymers and copolymers comprising phosphonate-containing monomeric groups are effective in inhibiting formation of chemical stains on dental surfaces. Furthermore, such polymers and copolymers exhibit improved compatibility with a halogenated diphenylether antibacterial agent, for example in presence of an antibacterial enhancing agent such as PVME/MA, by comparison with known polyphosphate-based anti-staining agents.

Accordingly there is now provided a dentifrice comprising:
(i) an anti-staining effective amount of an orally acceptable polymer or copolymer that comprises a plurality of monomeric groups of formula (I)

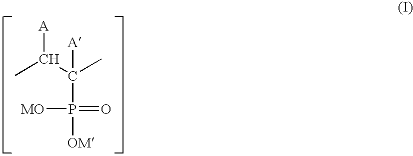

wherein:
(a) one of A and A′ is hydrogen and the other is a moiety (X)ₙ(R)ₘ,
(b) n in individual such moieties is independently 0 or 1,
(c) linking groups X if present independently comprise an oxygen, sulfur, nitrogen, phosphorus or silicon atom,
(d) where n is 0, m is 1, and where n is 1, m is independently an integer from 1 to 3 as determined by X,
(e) terminal groups R are independently hydrogen or C₁₋₁₈ organic radicals, and
(f) M and M′ are independently selected from hydrogen, alkali metal and ammonium;
said polymer or copolymer having an average molecular weight of at least about 1,000;
(ii) an antibacterial effective amount of an orally acceptable halogenated diphenylether antibacterial agent; and
(iii) an antibacterial enhancing effective amount of an orally acceptable PVME/MA copolymer.

Optionally one or more additional oral care agents, for example a cleaning agent such as an abrasive and/or a surfactant, can be included in the dentifrice.

In a further embodiment, there is provided a method of treating and/or preventing dental plaque and/or chemical stains on a dental surface, the method comprising applying a dentifrice as described above to the surface, for example by brushing.

An illustrative advantage of a dentifrice as provided herein is that it can be suitable for formulation as a single-component dentifrice, due to compatibility of the phosphonate-containing compound with the antibacterial agent. Where, by contrast, the antibacterial and anti-staining agents are incompatible, as in the case of a halogenated diphenylether antibacterial agent and a polyphosphate anti-staining agent, it is generally necessary to segregate these agents in separate components of the dentifrice, for example using a dual-chamber container, thereby incurring added cost and inconvenience.

Further advantages are obtainable with dentifrices representative of particular embodiments of the invention as pointed out below.

### DETAILED DESCRIPTION

A “chemical stain” herein is a discoloration of a dental surface caused by adsorption or absorption of a colored agent on or into the surface, or caused by chemical reaction of material of the dental surface (e.g., dental enamel) with a colored or noncolored agent contacting the surface. “Chemical staining” herein means formation and/or development of a chemical stain.

“Inhibition” of chemical staining as an object or result of treatment herein means reduction or prevention of stains that would otherwise form or develop subsequent to the time of the treatment. Such inhibition can range from a small but observable or measurable reduction to complete prevention of subsequent staining, by comparison with an untreated or placebo-treated dental surface.

A “dental surface” herein is a surface of a natural tooth or a hard surface of artificial dentition including a crown, cap, filling, bridge, dental implant and the like.

An “orally acceptable” compound or composition is one that is not harmful to a mammal in amounts disclosed herein when retained in the mouth, without swallowing, for a period sufficient to permit effective contact with a dental surface as required herein. In general, such a compound or composition is not harmful even if unintentionally swallowed.

“Average molecular weight” herein means a weight average as opposed to a number average, except where number average molecular weight is expressly stated. Weight average molecular weight (MW_{w}) can be determined, for example, by light scattering, small angle neutron scattering (SANS) or sedimentation velocity techniques. Number average molecular weight (MWₙ) can be determined, for example, by techniques involving gel permeation chromatography, osmometry, end-group titration or colligative properties.

Dentifrices include without limitation toothpastes, gels and powders.

The method of the invention is applicable to dental surfaces of nonhuman mammals such as companion animals (e.g., dogs and cats), as well as to humans. In one embodiment the dental surface is a surface of a natural tooth of a mammal, for example a human.

Where the dental surface is substantially free of chemical stains, the present method is effective to inhibit formation and development of new chemical stains, as can occur for example by oral use of tobacco products (including smoking) or by drinking tea or coffee, subsequent to treatment according to the method. Where the dental surface already possesses some degree of chemical staining, the present method is effective to inhibit further development of the existing stain. In some embodiments, for example where the dentifrice comprises a dental whitening agent such as a peroxide, the present method can remove, partially or completely, an existing chemical stain as well as inhibit subsequent staining.

In one embodiment the method further comprises, after applying the dentifrice to the dental surface, exposing the surface to a chemical stain inducing material such as a tobacco product, tea or coffee. Chemical staining resulting from such exposure is, in this embodiment, inhibited by the prior contacting of the dental surface with the dentifrice.

It is desirable according to the present method that the dental surface should be brushed with the dentifrice for a period sufficient to provide effective inhibition of chemical staining by the phosphonate-containing compound. Depending on various factors including the particular phosphonate-containing compound selected, other materials present in combination with the phosphonate-containing compound, and the desired degree and/or duration of inhibition of staining, a suitable minimum period of brushing is about 30 seconds to about 5 minutes, or in one embodiment at least about 1 minute, in another at least about 2 minutes.

Increasing the degree of agitation in the mouth during brushing can lead to improved contact of the phosphonate-containing compound with the dental surface and enhance the degree of inhibition of staining. Thus vigorous brushing can be particularly effective.

The phosphonate-containing compound, component (i) of the dentifrice, is a polymer or copolymer comprising a plurality of monomeric groups of formula (I) above. Such polymers and copolymers are illustratively disclosed in above-cited U.S. Pat. No. 5,032,386. In one embodiment the monomeric groups are recurring groups, i.e., a plurality of similar groups are present in the polymer or copolymer. In a particular embodiment, the phosphonate-containing compound is a homopolymer.

In one embodiment, A in the monomeric groups of formula (I) is a moiety (X)ₙ(R)ₘ as hereinabove defined, and A′ is hydrogen. In another embodiment, A is hydrogen and A′ is a moiety (X)ₙ(R)ₘ as hereinabove defined. According to either one of these embodiments, (X)ₙ(R)ₘ is illustratively selected from the group consisting of hydrogen; alkyl, cycloalkyl, alkenyl, acyl, alkoxy, alkylthio, alkylsulfoxy, alkylsulfonyl, alkylamino, dialkylamino, dialkylphosphinyl, dialkylphosphinoxy and trialkylsilyl radicals having up to 6 carbon atoms; and benzyl, benzoyl, benzyloxy, benzylthio, benzylsulfoxy, benzylsulfonyl, benzylamino, benzoylamido, phenyl, phenoxy, phenylthio, phenylsulfoxy, phenylsulfonyl, phenylamino, phenylacetamido, xylyl, pyridyl and furanyl radicals.

In one embodiment, n is 0 and R is selected from hydrogen, C₁₋₆ alkyl, C₃₋₆ cycloalkyl, phenyl and benzyl radicals.

Illustratively, the phosphonate-containing compound is a homopolymer wherein A in formula (I) is (X)ₙ(R)ₘ where n is 0, m is 1 and R is a C₁₋₆ alkyl or phenyl group, and A′ is hydrogen. Where R is methyl, such a homopolymer is poly(1-phosphonopropene) or a salt thereof. Alternatively, such a homopolymer where R is phenyl is poly(α-styrenephosphonic acid) or a salt thereof.

The phosphonate-containing compound can be present in its phosphonic acid form, where M and M′ are each hydrogen, or as a salt (including partial salt) thereof, wherein, in at least one monomer, at least one of M and M′ is alkali metal, typically sodium or potassium, or ammonium.

In one embodiment the phosphonate-containing compound is a homopolymer of vinylphosphonic acid, or a salt (including partial salt) thereof. Such a compound is described herein as a “polyvinylphosphonate” and can be prepared by any process known in the art, including processes disclosed in above-cited patents and publications.

Whether the phosphonate-containing compound is a polyvinylphosphonate or otherwise, it has an average molecular weight of at least about 1,000, typically about 1,000 to about 100,000 but optionally greater. In various embodiments the average molecular weight of the phosphonate-containing compound is about 5,000 to about 100,000, about 10,000 to about 100,000, about 15,000 to about 100,000, about 20,000 to about 100,000, about 25,000 to about 100,000 or about 25,000 to about 90,000. In one embodiment the average molecular weight is not less than about 22,000, for example about 22,000 to about 90,000, about 22,000 to about 70,000 or about 25,000 to about 35,000. In another embodiment the average molecular weight is not greater than about 30,000, for example about 3,500 to about 30,000 or about 6,000 to about 16,000. It will be noted that for a given polymer or copolymer, number average molecular weights are typically lower than the weight average molecular weights recited herein; for instance a polyvinylphosphonic acid having a weight average molecular weight (MW_{w}) of about 28,000 can have a number average molecular weight (MWₙ) of about 18,000.

A suitable amount of the phosphonate-containing compound present in the dentifrice depends on such factors as the particular compound selected, other materials present in the composition, and the desired degree and/or duration of inhibition of staining. Illustratively, whether the phosphonate-containing compound is a polyvinylphosphonate or otherwise, it is usefully present in the dentifrice at a concentration of about 0.1% to about 10% by weight, although greater or lesser concentrations can be useful in particular cases. In one embodiment, the composition comprises a polyvinylphosphonate at about 0.5% to about 5% by weight. Although phosphonate-containing compounds such as polyvinylphosphonic acid (PVPA) can be supplied as dispersions in water, amounts and concentrations are expressed herein on a dry matter (i.e., water-free) basis unless otherwise stated. Also unless otherwise stated, amounts and concentrations of polyvinylphosphonate salts are expressed herein on a PVPA equivalent basis.

The antibacterial agent, component (ii) of the dentifrice, is an orally acceptable halogenated diphenylether compound, for example 2,4,4′-trichloro-2′-hydroxydiphenylether (triclosan) or 2,2′-dihydroxy-5,5′-dibromodiphenylether and is present in an antibacterial effective amount, typically about 0.1% to about 10%, for example about 0.5% to about 5% by weight.

The antibacterial enhancing agent, component (iii) of the dentifrice, is an orally acceptable PVME/MA copolymer and is present in an antibacterial enhancing effective amount, typically about 0.1% to about 20%, for example about 0.5% to about 10% by weight. Generally the methyl vinyl ether to maleic anhydride ratio in the copolymer is about 1:4 to about 4:1, and the copolymer has an average molecular weight of about 30,000 to about 1,000,000, for example about 30,000 to about 500,000.

The orally acceptable vehicle of a composition useful according to the invention can comprise any oral care active(s) and/or carrier(s) known in the art, in addition to the components mentioned above. Classification herein of an ingredient as an active or a carrier ingredient is made for clarity and convenience, and no inference should be drawn that a particular ingredient necessarily functions in the composition in accordance with its classification herein.

Among useful oral care actives are those addressing, without limitation, appearance and structural changes to teeth, treatment and prevention of plaque, calculus, dental caries, cavities, abscesses, inflamed and/or bleeding gums, gingivitis, oral infective and/or inflammatory conditions in general, tooth sensitivity, halitosis and the like. Thus, among useful actives for optional inclusion in a composition useful according to the invention are whitening agents, anticalculus agents, fluoride ion sources, stannous ion sources, zinc ion sources, antimicrobial agents additional to the halogenated diphenylether antibacterial agent, antioxidants, sialagogues, breath freshening agents, antiplaque agents, anti-inflammatory agents, desensitizing agents, analgesics and nutrients. One active, or more than one active of the same or different classes, can optionally be present. Actives should be selected for compatibility with each other and with other ingredients of the composition.

In one embodiment the composition comprises, in addition to components (i), (ii) and (iii) above, at least one whitening agent. Any orally acceptable whitening agent can be used, including without limitation peroxy compounds, chlorine dioxide, chlorites and hypochlorites. For example, chlorites and hypochlorites of alkali and alkaline earth metals such as lithium, potassium, sodium, magnesium, calcium and barium can be used. Alternatively or in addition, one or more peroxy compounds can be used. Peroxy compounds include hydrogen peroxide, peroxides of alkali and alkaline earth metals, organic peroxy compounds and peroxy acids and salts thereof. Any orally acceptable compound that delivers a perhydroxy (—OOH⁻) ion is useful.

Peroxides of alkali and alkaline earth metals include lithium peroxide, potassium peroxide, sodium peroxide, magnesium peroxide, calcium peroxide and barium peroxide.

Organic peroxy compounds include, for example, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like.

Peroxy acids and their salts include organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium.

One or more whitening agents are optionally present in a tooth-whitening effective total amount, typically about 0.1% to about 90%, for example about 0.5% to about 50% or about 1% to about 30% by weight of the composition. Where peroxy compounds such as hydrogen peroxide are included, they can suitably be present in a total hydrogen peroxide equivalent amount of about 0.5% to about 50%, for example about 1% to about 30% by weight of the composition. Peroxy compounds can illustratively be present in a total hydrogen peroxide equivalent amount of about 2% to about 10% by weight in a dentifrice composition, or about 10% to about 30% by weight in a liquid whitener composition.

Peroxy compounds are typically incompatible with halogenated diphenylether antibacterial agents such as triclosan, thus if a peroxy compound is included as a whitening agent in the composition it should be segregated from the antibacterial agent, for example by use of a dual-chamber dispensing container, by encapsulation or by some other means.

In a further embodiment a composition useful according to the invention comprises, in addition to components (i), (ii) and (iii) above, at least one anticalculus agent. Any orally acceptable anticalculus agent can be used, including without limitation phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts illustratively include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and the like, wherein sodium can optionally be replaced by potassium or ammonium. As noted above, however, polyphosphates tend to be incompatible with halogenated diphenylether antibacterial agents, thus if a polyphosphate is included as an anticalculus agent in the composition it should be segregated from the antibacterial agent, for example by a means as indicated above. It is further noted that the PVME/MA copolymer present as component (iii) of the composition can provide useful anticalculus activity in addition to serving as an antibacterial enhancing agent.

One or more anticalculus agents are optionally present in an anticalculus effective total amount, typically about 0.01% to about 50%, for example about 0.05% to about 25% or about 0.1% to about 15% by weight of the composition.

In a still further embodiment a composition useful according to the invention comprises, in addition to components (i), (ii) and (iii) above, at least one fluoride ion source useful, for example, as an anti-caries agent. Any orally acceptable fluoride ion source can be used, including without limitation potassium, sodium and ammonium fluorides and monofluorophosphates, stannous fluoride, indium fluoride and the like. Water-soluble fluoride ion sources are typically used. One or more fluoride ion sources are optionally present in an amount providing a total of about 0.0025% to about 2%, for example about 0.005% to about 1% or about 0.01% to about 0.3%, of fluoride ions by weight of the composition.

In a still further embodiment a composition useful according to the invention comprises, in addition to components (i), (ii) and (iii) above, at least one stannous ion source useful, for example, in helping reduce gingivitis, plaque, caries or sensitivity. Any orally acceptable stannous ion source can be used, including without limitation stannous fluoride, other stannous halides such as stannous chloride dihydrate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5% by weight of the composition.

In a still further embodiment a composition useful according to the invention comprises, in addition to components (i), (ii) and (iii) above, at least one zinc ion source useful, for example, as an antimicrobial, anticalculus or breath-freshening agent. Any orally acceptable zinc ion source can be used, including without limitation zinc citrate, zinc sulfate, zinc glycinate, sodium zinc citrate and the like. One or more zinc ion sources are optionally and illustratively present in a total amount of about 0.05% to about 3%, for example about 0.1% to about 1%, by weight of the composition.

In a still further embodiment a composition useful according to the invention comprises, in addition to components (i), (ii) and (iii) above, at least one antimicrobial (e.g., antibacterial) agent other than a halogenated diphenylether. Any orally acceptable such antimicrobial agent can be used, including without limitation 8-hydroxyquinoline and salts thereof, copper (II) compounds such as copper (II) chloride, fluoride, sulfate and hydroxide, phthalic acid and salts thereof such as magnesium monopotassium phthalate, chlorhexidine, alexidine, hexetidine, sanguinarine, benzalkonium chloride, salicyanilide, domiphen bromide, alkylpyridinium chlorides such as cetylpyridinium chloride (CPC) (including combinations of CPC with zinc and/or enzymes), tetradecylpyridinium chloride and N-tetradecyl-4-ethylpyridinium chloride, octenidene, iodine, sulfonamides, bisbiguanides, phenolics, piperidino derivatives such as delmopinol and octapinol, zinc ion sources, magnolia extract, grapeseed extract, phenol, thymol, eugenol, menthol, geraniol, carvacrol, citral, eucalyptol, catechol, 4-allylcatechol, hexyl resorcinol, 2,2′-methylene bis(4-chloro-6-bromophenol), methyl salicylate, antibiotics such as augmentin, amoxicillin, tetracycline, doxycycline, minocycline, metronidazole, neomycin, kanamycin and clindamycin, and the like. One or more antimicrobial agents other than a halogenated diphenylether are optionally present together with the halogenated diphenylether (component (ii) of the composition) in an antimicrobial effective total amount.

In a still further embodiment a composition useful according to the invention comprises, in addition to components (i), (ii) and (iii) above, at least one antioxidant. Any orally acceptable antioxidant can be used, including without limitation butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin and the like. One or more antioxidants are optionally present in an antioxidant effective total amount.

In a still further embodiment a composition useful according to the invention comprises, in addition to components (i), (ii) and (iii) above, a sialagogue (saliva stimulating agent), useful for example in amelioration of dry mouth. Any orally acceptable sialagogue can be used, including without limitation food acids such as citric, lactic, malic, succinic, ascorbic, adipic, fumaric and tartaric acids. One or more sialagogues are optionally present in the composition in a saliva stimulating effective total amount.

In a still further embodiment a composition useful according to the invention comprises, in addition to components (i), (ii) and (iii) above, a breath freshening agent. Any orally acceptable breath freshening agent can be used, including without limitation zinc salts such as zinc gluconate, zinc citrate and zinc chlorite, α-ionone and the like. One or more breath freshening agents are optionally present in a breath freshening effective total amount.

In a still further embodiment a composition useful according to the invention comprises, in addition to components (i), (ii) and (iii) above, an antiplaque, including plaque disrupting, agent. Any orally acceptable antiplaque agent can be used, including without limitation stannous, copper, magnesium and strontium salts, dimethicone copolyols such as cetyl dimethicone copolyol, papain, glucoamylase and glucose oxidase. One or more antiplaque agents are optionally present in an antiplaque effective total amount.

In a still further embodiment a composition useful according to the invention comprises, in addition to components (i), (ii) and (iii) above, at least one anti-inflammatory agent. Any orally acceptable anti-inflammatory agent can be used, including without limitation steroidal agents such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone and phenylbutazone. One or more anti-inflammatory agents are optionally present in an anti-inflammatory effective amount.

In a still further embodiment a composition useful according to the invention comprises, in addition to components (i), (ii) and (iii) above, at least one desensitizing agent. Potassium salts such as potassium nitrate are illustratively useful in this regard, as is sodium nitrate. Alternatively or in addition a local or systemic analgesic such as aspirin, codeine, acetaminophen, sodium salicylate or triethanolamine salicylate can be used. One or more desensitizing agents and/or analgesics are optionally present in a desensitizing and/or analgesic effective amount.

In a still further embodiment a composition useful according to the invention comprises, in addition to components (i), (ii) and (iii) above, at least one nutrient. Suitable nutrients include vitamins, minerals and amino acids.

Among useful carriers for optional inclusion in a composition useful according to the invention are diluents, abrasives, bicarbonate salts, pH modifying agents, surfactants, foam modulators, thickening agents, viscosity modifiers, humectants, sweeteners, flavorants and colorants. One carrier material, or more than one carrier material of the same or different classes, can optionally be present. Carriers should be selected for compatibility with each other and with other ingredients of the composition.

In one embodiment a composition useful according to the invention comprises, in addition to components (i), (ii) and (iii) above, at least one diluent, for example water.

In a further embodiment a composition useful according to the invention comprises, in addition to components (i), (ii) and (iii) above, at least one abrasive, useful for example as a polishing agent. Any orally acceptable abrasive can be used, but type, fineness (particle size) and amount of abrasive should be selected so that tooth enamel is not excessively abraded in normal use of the composition. Suitable abrasives include without limitation silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. One or more abrasives are optionally present in an abrasive effective total amount, typically about 5% to about 70%, for example about 10% to about 50% or about 15% to about 30% by weight of the composition. Average particle size of an abrasive, if present, is generally about 0.1 to about 30 μm, for example about 1 to about 20 μm or about 5 to about 15 μm.

In a particular embodiment the composition comprises one or more high-cleaning silicas (HCS) to enhance whitening performance of the dentifrice by mechanically removing existing stain and debris from a dental surface by means of the HCS while inhibiting further accumulation of chemical stain by means of the phosphonate-containing compound, e.g., PVPA.

In a still further embodiment a composition useful according to the invention comprises, in addition to components (i), (ii) and (iii) above, at least one bicarbonate salt, useful for example to impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate and the like. One or more bicarbonate salts are optionally present in a total amount of 0.1% to about 50%, for example about 1% to about 20% by weight of the composition.

In a still further embodiment a composition useful according to the invention comprises, in addition to components (i), (ii) and (iii) above, at least one pH modifying agent. Such agents include acidifying agents to lower pH, basifying agents to raise pH and buffering agents to control pH within a desired range. For example, one or more compounds selected from acidifying, basifying and buffering agents can be included to provide a pH of about 2 to about 10, or in various illustrative embodiments about 2 to about 8, about 3 to about 9, about 4 to about 8, about 5 to about 7, about 6 to about 10, about 7 to about 9, etc. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and the like. One or more pH modifying agents are optionally present in a total amount effective to maintain the composition in an orally acceptable pH range.

In a still further embodiment a composition useful according to the invention comprises, in addition to components (i), (ii) and (iii) above, at least one surfactant, useful for example to compatibilize other components of the composition and thereby provide enhanced stability, to help in cleaning the dental surface through detergency, and to provide foam upon agitation, e.g., during brushing. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium coconut monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C₈₋₂₀ aliphatic secondary and tertiary amines having an anionic group such as carboxylate, sulfate, sulfonate, phosphate or phosphonate. A suitable example is cocoamidopropyl betaine. One or more surfactants are optionally present in a total amount of about 0.01% to about 10%, for example about 0.05% to about 5% or about 0.1% to about 2% by weight of the composition.

In a particular embodiment the composition comprises one or more surfactants, e.g., sodium lauryl sulfate, providing cleaning action. According to this embodiment, the phosphonate-containing compound, e.g., PVPA, can further enhance cleaning action provided by the surfactant alone.

In a still further embodiment a composition useful according to the invention comprises, in addition to components (i), (ii) and (iii) above, at least one foam modulator, useful for example to increase amount, thickness or stability of foam generated by the composition upon agitation. Any orally acceptable foam modulator can be used, including without limitation polyethylene glycols (PEGs), also known as polyoxyethylenes. High molecular weight PEGs are suitable, including those having an average molecular weight of about 200,000 to about 7,000,000, for example about 500,000 to about 5,000,000 or about 1,000,000 to about 2,500,000. One or more PEGs are optionally present in a total amount of about 0.1% to about 10%, for example about 0.2% to about 5% or about 0.25% to about 2% by weight of the composition.

In a still further embodiment a composition useful according to the invention comprises, in addition to components (i), (ii) and (iii) above, at least one thickening agent, useful for example to impart a desired consistency and/or mouth feel to the composition. Any orally acceptable thickening agent can be used, including without limitation carbomers, also known as carboxyvinyl polymers, carrageenans, also known as Irish moss and more particularly τ-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. One or more thickening agents are optionally present in a total amount of about 0.01% to about 15%, for example about 0.1% to about 10% or about 0.2% to about 5% by weight of the composition.

In a still further embodiment a composition useful according to the invention comprises, in addition to components (i), (ii) and (iii) above, at least one viscosity modifier, useful for example to inhibit settling or separation of ingredients or to promote redispersibility upon agitation of a liquid composition. Any orally acceptable viscosity modifier can be used, including without limitation mineral oil, petrolatum, clays and organomodified clays, silica and the like. One or more viscosity modifiers are optionally present in a total amount of about 0.01% to about 10%, for example about 0.1% to about 5% by weight of the composition.

In a still further embodiment a composition useful according to the invention comprises, in addition to components (i), (ii) and (iii) above, at least one humectant, useful for example to prevent hardening of a toothpaste upon exposure to air. Any orally acceptable humectant can be used, including without limitation polyhydric alcohols such as glycerin, sorbitol, xylitol or low molecular weight PEGs. Most humectants also function as sweeteners. One or more humectants are optionally present in a total amount of about 1% to about 50%, for example about 2% to about 25% or about 5% to about 15% by weight of the composition.

In a still further embodiment a composition useful according to the invention comprises, in addition to components (i), (ii) and (iii) above, at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like. One or more sweeteners are optionally present in a total amount depending strongly on the particular sweetener(s) selected, but typically about 0.005% to about 5% by weight of the composition.

In a still further embodiment a composition useful according to the invention comprises, in addition to components (i), (ii) and (iii) above, at least one flavorant, useful for example to enhance taste of the composition. Any orally acceptable natural or synthetic flavorant can be used, including without limitation vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or warming effects. Such ingredients illustratively include menthol, menthyl acetate, menthyl lactate, camphor, eucalyptus oil, eucalyptol, anethole, eugenol, cassia, oxanone, α-irisone, propenyl guaiethol, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of about 0.01% to about 5%, for example about 0.1% to about 2.5% by weight of the composition.

In a still further embodiment a composition useful according to the invention comprises, in addition to components (i), (ii) and (iii) above, at least one colorant. Colorants herein include pigments, dyes, lakes and agents imparting a particular luster or reflectivity such as pearling agents. A colorant can serve a number of functions, including for example to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride and the like. One or more colorants are optionally present in a total amount of about 0.001% to about 20%, for example about 0.01% to about 10% or about 0.1% to about 5% by weight of the composition.

In particular illustrative embodiments, a dentifrice composition of the invention comprises, in addition to PVPA, triclosan and PVME/MA, one or more of the following ingredients:

hydrated silica;

glycerin;

carrageenan;

sorbitol;

sodium CMC;

sodium fluoride;

sodium lauryl sulfate;

sodium saccharin; and

titanium dioxide.

Degree of staining or stain inhibition on a dental surface can be observed visually, for example with the aid of color comparison charts, gauges or shade guides, e.g., as described by Browning (2003). Journal of Esthetic Restorative Dentistry 15 Supp. 1, S13-S20, incorporated herein by reference.

Alternatively, staining or inhibition thereof can be measured by colorimetry, using any suitable instrument such as a Minolta Chromameter, e.g., model CR-321 (Minolta Corp., Ramsey, N.J.). The instrument can be programmed, for example, to measure Hunter Lab values or L*a*b* values according to the standard established by the International Committee of Illumination (CIE). The L*a*b* system provides a numerical representation of three-dimensional color space where L* represents a lightness axis, a* represents a red-green axis and b* represents a yellow-blue axis. The L* and b* axes are typically of greatest applicability to tooth stain inhibition, which can be measured as increase in whiteness relative to an untreated surface. Increase in whiteness can be computed from differences in L*, a* and b* values between untreated and treated surfaces. A useful parameter is ΔE*, calculated as the square root of the sum of the squares of differences in L*, a* and b* values, using the formula:

ΔE*=[(ΔL*)²+(Δa*)²+(Δb*)²]^{1/2}

A higher value of ΔE* indicates greater increase in whiteness.

Evaluation of effectiveness of stain inhibition treatments of the invention can be made, for example, in clinical studies using human volunteers, or in vivo in animals, conducted according to appropriate protocols.

Suitable in vitro protocols are also available for evaluation of stain inhibition treatments, including those described in Examples herein and in published literature. See for example Stookey et al. (1982), Journal of Dental Research 61(11), 1236-1239, and Rice et al. (2001), Journal of Clinical Dentistry 12(2), 34-37, both incorporated herein by reference.

The invention can further be understood by reference to the following nonlimiting example.

### EXAMPLE

Toothpaste compositions were prepared having ingredients as shown in Table 1. The compositions were similar except for the presence or absence of PVPA. The glycerin, carboxymethylcellulose sodium, propylene glycol and τ-carrageenan were mixed together for at least about 5 minutes. The sorbitol, water, titanium dioxide, sodium saccharin and sodium fluoride were then added and the resulting mixture was heated to 60-71° C. with mixing for at least about 15 minutes. The Gantrez™ S-97 (PVME/MA), PVPA (if included) and sodium hydroxide were then added with mixing for at least about 5 minutes. The hydrated silica was then added and mixing continued for at least about 15 minutes under vacuum. Finally, the triclosan, sodium lauryl sulfate and flavorant were added and mixing continued under vacuum for at least a further 10 minutes.

TABLE 1
Composition of toothpastes with and without PVPA

Weight %

| Ingredient | −PVPA | +PVPA |
|---|---|---|
| water | 15.28 | 11.49 |
| sodium fluoride | 0.24 | 0.24 |
| sodium saccharin | 0.30 | 0.30 |
| glycerin | 20.00 | 20.00 |
| sodium CMC | 1.10 | 1.10 |
| propylene glycol | 0.50 | 0.50 |
| [symbol] -carrageenan | 0.40 | 0.40 |
| sorbitol, 70% in water | 20.85 | 20.85 |
| titanium dioxide | 0.75 | 0.75 |
| Gantrez ™ S-97, 13% in water | 15.00 | 15.00 |
| PVPA, 32% in water | 0.00 | 2.99 |
| sodium hydroxide, 50% in water | 1.20 | 2.00 |
| hydrated silicas | 21.50 | 21.50 |
| triclosan | 0.30 | 0.30 |
| flavor | 1.00 | 1.00 |
| sodium lauryl sulfate powder | 1.50 | 1.50 |

The PVPA used in this example had a weight average molecular weight of 8,000.

To verify that antibacterial activity of the triclosan, as enhanced by PVME/MA, was not adversely affected by PVPA, the compositions of Example 1 with and without PVPA were each compared with a standard toothpaste (Colgate® Dental Cream) for antibacterial effect using a chemostat as described for example by Herles et al. (1994), J. Dent. Res. 73(11), 1748-1755, incorporated herein by reference. Results are shown in Table 2.

TABLE 2
Antibacterial efficacy of toothpastes with and without PVPA

| Toothpaste | % Bacterial Reduction |
|---|---|
| −PVPA | 36.7 |
| +PVPA | 46.3 |

The difference in antibacterial activity shown in Table 2 was not statistically significant. The data show that adding PVPA to a dentifrice containing triclosan and PVME/MA does not impair the antibacterial action of the dentifrice.

Relative effectiveness of the compositions of this example with and without PVPA in inhibition of staining of a dental surface, and in cleaning of a stained dental surface, was determined by the following procedure, adapted from Baig et al. (2002), op. cit.
1. Human saliva, kept on ice until needed, was centrifuged at 10,000 rpm for 10 minutes at room temperature. The supernatant was collected and kept on ice until needed.
2. Disks of synthetic hydroxyapatite (SHAP, to simulate a natural dental surface) were rinsed in water, blotted and allowed to air-dry. Their color parameters on the L*a*b* system as established by CIE was measured using a Minolta CR-321 chromameter.
3. The SHAP disks were then placed in a 17×100 mm polystyrene test tube, one disk per tube, and 2 ml of saliva supernatant was added to each disk. The test tubes were incubated in a shaker bath at 37° C. overnight.
4. The disks were removed from the saliva supernatant, rinsed in water and blotted dry, and were then returned to the test tubes.
5. A slurry was prepared of the toothpaste composition at a 1:10 dilution in water, and 2 ml of the slurry was added to each disk, followed by incubation in the shaker bath at 37° C. for 5 minutes.
6. The disks were removed from the toothpaste slurry, rinsed in water and blotted dry, and were then returned to the test tubes.
7. A staining cycle was then applied to the disks, each step in the cycle involving incubation in the shaker bath at 37° C. for the time period shown, followed by washing three times with water:

| | |
|---|---|
| saliva | 20 minutes |
| 0.12% chlorhexidine rinse (Periogard ®) | 1 minute |
| saliva | 20 minutes |
| instant coffee | 15 minutes |

8. The staining cycle was repeated for a total of three cycles.
9. A further 2 ml of the toothpaste slurry was added to each disk, followed by incubation in the shaker bath at 37° C. for 5 minutes.
10. The disks were rinsed in water, blotted dry and allowed to air dry. A further measurement of color parameters was obtained.

Inhibition of chemical staining was determined as ΔL* and ΔE*, in each case measuring the difference before and after the entire procedure described above. ΔL* was reported as an absolute value (i.e., a reduction in the value of L* was reported as a positive number). A lower value of ΔL* (absolute) and ΔE* is indicative of greater inhibition of staining. i.e. greater stain resistance of the treated surface and thus enhanced anti-staining performance of the toothpaste. Results are shown in Table 3.

TABLE 3
Anti-staining performance of toothpastes with and without PVPA

| Toothpaste | ΔL* | ΔE* |
|---|---|---|
| −PVPA | 19.18 | 20.87 |
| +PVPA | 7.64 | 8.37 |

The toothpaste containing PVPA exhibited significantly lower values of ΔL* (absolute) and ΔE* than the comparative toothpaste lacking PVPA. This result demonstrates a high degree of effectiveness of PVPA as a toothpaste ingredient in inhibiting staining of dental surfaces.

Chemical cleaning action was determined as ΔL* and ΔE*, in each case measuring the difference before and after the post-staining toothpaste treatment as described above. A higher value of ΔL* and ΔE* is indicative of greater chemical cleaning action of the toothpaste. Results are shown in Table 4.

TABLE 3
Chemical cleaning action of toothpastes with and without PVPA

| Toothpaste | ΔL* | ΔE* |
|---|---|---|
| water (control) | 1.90 | 5.00 |
| −PVPA | 9.06 | 13.02 |
| +PVPA | 15.67 | 20.23 |

The toothpaste containing PVPA exhibited significantly higher values of ΔL* and ΔE* than the comparative toothpaste lacking PVPA. This result demonstrates that PVPA enhances the cleaning action of a toothpaste containing sodium lauryl sulfate as a surfactant.

What is claimed is:

1. A dentifrice composition comprising
(i) an anti-staining effective amount of an orally acceptable phosphonate-containing polymer or copolymer that comprises a plurality of monomeric groups of formula 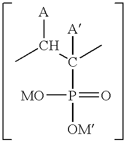
wherein:
(a) one of A and A′ is hydrogen and the other is a moiety (X)ₙ(R)ₘ,
(b) n in individual such moieties is independently 0 or 1.
(c) linking groups X if present independently comprise an oxygen, sulfur, nitrogen, phosphorus or silicon atom,
(d) where n is 0, m is 1, and where n is 1, m is independently an integer from 1 to 3 as determined by X,
(e) terminal groups R are independently hydrogen or C-18 organic radicals, and
(l) M and M′ are independently selected from hydrogen, alkali metal and ammonium;
said polymer or copolymer having an average molecular weight of at least about 1,000;
(ii) an antibacterial effective amount of an orally acceptable halogenated diphenylether antibacterial agent; and
(iii) an antibacterial enhancing effective amount of an orally acceptable polyvinylmethylether/maleic anhydride copolymer.

2. The composition of claim 1 wherein the phosphonate-containing compound has an average molecular weight of about 1,000 to about 100,000.

3. The composition of claim 1 wherein the phosphonate-containing compound has an average molecular weight of about 3,500 to about 30,000.

4. The composition of claim 1 wherein the phosphonate-containing compound has an average molecular weight of about 6,000 to about 16,000.

5. The composition of claim 1 wherein the phosphonate-containing polymer or copolymer is a polyvinylphosphonate that is a homopolymer of vinylphosphonic acid, or a salt or partial salt thereof.

6. The composition of claim 5 that comprises said polyvinylphosphonate in a polyvinylphosphonic acid equivalent amount of about 0.1% to about 10% by weight.

7. The composition of claim 5 that comprises said polyvinylphosphonate in a polyvinylphosphonic acid equivalent amount of about 0.5% to about 5% by weight.

8. The composition of claim 1 wherein the halogenated diphenylether compound is triclosan.

9. The composition of claim 1 wherein the halogenated diphenylether compound is present in an amount of about 0.1% to about 10% by weight.

10. The composition of claim 1 wherein the halogenated diphenylether compound is present in an amount of about 0.5% to about 5% by weight.

11. The composition of claim 1 wherein the polyvinylmethylether/maleic anhydride copolymer has a methyl vinyl ether to maleic anhydride ratio of about 1:4 to about 4:1.

12. The composition of claim 1 wherein the polyvinylmethylether/maleic anhydride copolymer has an average molecular weight of about 30,000 to about 1,000,000.

13. The composition of claim 1 wherein the polyvinylmethylether/maleic anhydride copolymer is present in an amount of about 0.1% to about 20% by weight.

14. The composition of claim 1 wherein the polyvinylmethylether/maleic anhydride copolymer is present in an amount of about 0.5% to about 10% by weight.

15. The composition of claim 1, further comprising at least one ingredient selected from the group consisting of whitening agents, anticalculus agents, fluoride ion sources, stannous ion sources, zinc ion sources, antimicrobial agents additional to the halogenated diphenylether antibacterial agent, antioxidants, sialagogues, breath freshening agents, antiplaque agents, anti-inflammatory agents, desensitizing agents, analgesics, nutrients, diluents, abrasives, bicarbonate salts, pH modifying agents, surfactants, foam modulators, thickening agents, viscosity modifiers, humectants, sweeteners, flavorants and colorants.

16. The composition of claim 1, further comprising a high-cleaning silica.

17. The composition of claim 1, further comprising sodium lauryl sulfate.

18. The composition of claim 1 that is a toothpaste.

19. A method of treating and/or preventing dental plaque and/or chemical stains on a dental surface, the method comprising applying the dentifrice composition of claim 1 to the surface.

20. The method of claim 19 wherein the dentifrice composition is applied to the dental surface by brushing.
